(19) United States
(12) Patent Application Publication
(10) Pub. No.: US 2013/0064849 A1
(43) Pub. Date: Mar. 14, 2013

(54) METHODS TO TREAT AND PREVENT CARDIOVASCULAR DISEASE USING HUMAN PAPILLOMAVIRUS VACCINES

(71) Applicants: Ken Fujise, Galveston, TX (US); Hsu-Ko Kuo, Friendswood, TX (US)

(72) Inventors: Ken Fujise, Galveston, TX (US); Hsu-Ko Kuo, Friendswood, TX (US)

(73) Assignee: THE BOARD OF REGENTS OF THE UNIVERSITY OF TEXAS, Austin, TX (US)

(21) Appl. No.: 13/548,854

(22) Filed: Jul. 13, 2012

Related U.S. Application Data
(60) Provisional application No. 61/572,325, filed on Jul. 14, 2011.

(51) Int. Cl. A61K 39/12; A61P 13/12; A61P 9/04; A61P 9/08; A61P 9/00; A61P 9/10

(52) U.S. Cl. 424/204.1

(57) ABSTRACT

Embodiments of the invention include methods of treating or preventing cardiovascular disease, including atherosclerosis, myocardial infarction, and stroke, by administering an HPV vaccine, particular a vaccine that induces immunity against an oncogenic HPV type such as types 16 and 18.

### STATEMENT REGARDING PRIORITY

This application claims priority to U.S. Provisional Patent Application No. 61/572,325 filed Jul. 14, 2011, which is incorporated herein by reference in its entirety.

### STATEMENT REGARDING FEDERALLY FUNDED RESEARCH

None.

### BACKGROUND

Research indicates that cigarette smoking, diabetes, dyslipidemia, and hypertension, collectively labeled as conventional risk factors, are important contributors to cardiovascular disease (CVD) (Stamler et al., Intervention Trial. Diabetes Care. 16(2):434-44, 1993; Verschuren et al. JAMA 274(2): 131-6, 1995; MacMahon et al. Lancet 335(8692):765-74, 1990) and that treatment of these risk factors is associated with reduced risk of future cardiovascular events (Collins et al. Lancet 335(8693):827-38, 1990). However, nearly 20% of individuals with CVD do not have any of the conventional risk factors (Khot et al. JAMA 290(7):898-904, 2003), indicating that other “nontraditional” factors may play an essential role in the development of CVD. There remains a need for methods of identifying and treating subjects in need of prophylactic and/or therapeutic intervention for CVD.

### SUMMARY

Certain embodiments are directed to methods for treating or preventing cardiovascular disease (CVD) in a subject comprising administering an HPV vaccine to a subject. In certain embodiments the subject is at least 27 years old. In a further embodiment, the subject is at least 25, 26, 27, 28, 29, 30, 35, 40, 45, 50, 55, 60, or 65 years old, including all ages and ranges there between. In certain aspects the subject is 45 to 55 years old. In certain aspects, the subject is female. In a further aspect a female subject has undergone menopause, a hysterectomy, or is otherwise sterile. In other aspects the subject is male. In certain aspects, a male subject has had a vasectomy or is otherwise sterile. The methods described herein can reduce the likelihood or risk of developing CVD or its sequelae.

The term “treatment” refers to the medical management of a patient with the intent to cure, ameliorate, or stabilize a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) inhibiting the disease, i.e., arresting its development; or (ii) relieving the disease, i.e., causing regression of the disease. In one aspect, the subject is a mammal such as a primate, and, in a further aspect, the subject is a human.

The terms “prevent,” “preventing” and “prevention” refer to the prevention of the onset of a disease or disorder, or of one or more symptoms thereof. In certain embodiments, the terms refer to the administration of a vaccine as provided herein, with or without other additional active compounds, prior to the onset of symptoms, particularly to patients at risk of CVD or disorders provided herein. The terms encompass the inhibition of a symptom of the particular disease. Patients with familial history of a disease in particular are candidates for preventive regimens in certain embodiments. In this regard, the term “prevention” may be interchangeably used with the term “prophylactic treatment.”

In certain aspects the HPV vaccine induces immunity to an oncogenic HPV. An oncogenic HPV can be HPV16, 18, 31, 33, 35, 39, 45, 51, 52, 56, 58, 59, and 68. In certain aspects, the HPV vaccine induces immunity to HPV16, HPV18, or both HPV16 and HPV 18. In certain aspects, the HPV vaccine induces an immune response to one or more of L1, L2, E1, E2, E3, E4, E5, E6, E7 or E8 proteins of HPV. In certain aspects, the HPV vaccine induces an immune response at least to a HPV L1 protein.

In certain aspects, the CVD is atherosclerosis, unstable angina, chronic stable angina, sudden cardiac death, aneurysm formation, intermittent claudication, renal artery stenosis, coronary heart disease, cerebrovascular disease, aorto-iliac disease, or peripheral vascular disease. In a further aspect, CVD is atherosclerosis. Subjects with or at risk of CVD may develop a number of complications or sequelae, including, but not limited to, myocardial infarction, stroke, angina pectoris, transient ischemic attacks, congestive heart failure, and aortic aneurysm. The treatment of CVD can reduce the risk of or prevent the occurrence of CVD sequelae.

In certain aspects, the subject can have one or more of the following CVD risk factors: hypertension, a family history of premature CVD, smoking, high total cholesterol, low HDL cholesterol, obesity, or diabetes.

In a further aspect, the methods can further comprise detecting the presence or absence of HPV in the subject prior to administering the HPV vaccine.

The term “providing” is used according to its ordinary meaning “to supply or furnish for use.” In some embodiments, the protein is provided directly by administering the protein, while in other embodiments, the protein is effectively provided by administering a nucleic acid that encodes the protein. In certain aspects the invention contemplates compositions comprising various combinations of nucleic acid, antigens, peptides, and/or epitopes.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens, cells or tissues infected with pathogens, or, in cases of autoimmunity or pathological inflammation, normal human cells or tissues.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION

Human papillomavirus (HPV) infection is one of the most common sexually transmitted infections in the United States. A recent study from the National Health and Nutrition Examination Survey (NHANES) reported an overall HPV prevalence of 26.8% (Dunne et al. JAMA 297(8):813-9, 2007). HPVs, especially “oncogenic” or cancer-associated types, are implicated as a causative agent of anogenital cancers (Munoz et al. N Engl J Med 348(6):518-27, 2003; Bjorge et al. Br J Cancer 87(1):61-4, 2002). The viral oncoproteins bind to cellular tumor-suppressor protein p53 and therefore induce the degradation of p53 through the ubiquitin pathway (Scheffner et al. Cell 63(6):1129-36, 1990; Werness et al. Science 248(4951):76-9, 1990). p53 is essential in regulating the process of atherosclerosis (Tabas, Circ Res. 88(8):747-9, 2001). Absence of p53 has been shown to accelerate atherosclerosis in vivo (Guevara et al. Nat Med 5(3):335-9, 1999) and human study also demonstrated an association between p53 inactivation and postatherectomy coronary restenosis (Speir et al. Science 265(5170):391-4, 1994). Although the role of HPV is well known in the development of anogenital cancers, the potential atherosclerotic outcome of HPV infection (through interaction between HPV oncoproteins and p53) is largely unknown and has not been investigated. In certain aspects, HPV infection is associated with CVD among women.

Based on the studies described herein, HPV infection is associated with myocardial infarction or stroke among U.S. women aged from 20 to 59. Cancer-associated HPV genotypes are especially related to the presence of CVD. The association between HPV and CVD still perseveres after adjustment for health/sex behaviors, medical conditions, cardiovascular risk burden and management, indicating that conventional risk factors cannot fully explain the relation of HPV to CVD and that presence of HPV infection, especially cancer-associated genotypes, is a strong and independent correlate for CVD. Example 1 describes the association between HPV infection and CVD by using a geographically dispersed and ethnically diverse representative sample of community-dwelling women living in the United States. Potential confounders were also comprehensively considered.

Without being bound by theory, it is believed that the products of two tumor suppressor genes, p53 and retinoblastoma protein (pRb), can be involved in the mechanisms for the association between HPV and CVD. HPV oncoproteins interact with and inactivate both p53 and pRb. In animal studies using different models, knockout of p53 gene (Guevara et al. Nat Med 5(3):335-9, 1999), transplanting mice with bone marrow from p53 knockout mice (van Vlijmen et al. Circ Res 88(8):780-6, 2001), and macrophage-specific loss of p53 function (Merched et al. Arterioscler Thromb Vasc Biol 23(9):1608-14, 2003) were all associated with considerably increased atherosclerotic lesion size with the presence of more lesion necrosis (van Vlijmen et al. Circ Res 88(8):780-6, 2001), decreased apoptotic activity (van Vlijmen et al. Circ Res 88(8):780-6, 2001), and extensive cellular proliferation (Guevara et al. Nat Med 5(3):335-9, 1999; Merched et al. Arterioscler Thromb Vasc Biol 23(9):1608-14, 2003) in the atherosclerotic lesions. Retinoblastoma gene, the first tumor suppressor gene identified molecularly, also plays a pivotal role in regulating cell proliferation. In fact, introduction of Rb gene via an adenovirus vector has been shown to inhibit vascular smooth muscle cell (VSMC) proliferation (Li et al. Chin Med J (Engl) 110(12):950-4, 1997). One animal study showed retinoblastoma gene deleted mice had enhanced atherosclerosis development, involving increase in atherosclerotic lesion area and increase in smooth muscle cell area (Boesten et al. FASEB J. 20(7):953-5, 2006). Taken together, HPV oncoproteins inactivate tumor suppressor gene products (p53 and pRb) and may therefore accelerate atherosclerotic process.

Embodiments of the invention identify HPV as having a role in the management and prevention of CVD. Aspects of the invention can serve as a theoretical basis for additional benefit in cardiovascular health with HPV vaccination. Bivalent HPV vaccine (HPV2; Cervarix™), directed against two oncogenic types HPV 16 and 18, and quadrivalent HPV vaccine (HPV 4; Gardasil™), directed against two oncogenic types HPV 16 and 18 as well as two nononcogenic types HPV 6 and 11, are the current vaccines to prevent HPV infection and related STDs and tumors. Although HPV 16 and 18 are only two cancer-associated oncogenic vaccine types, efficacy of vaccines on protection against cervical lesions due to other nonvaccine oncogenic types (HPV 31, 33, 35, 39, 45, 51, 52, 56, 58, 59, 66, and 68) was demonstrated and estimated to be 37.4% (Food and Drug Administration. Product approval-prescribing information, Cervarix™ [human papillomavirus bivalent (types 16 and 18) vaccine, recombinant], GlaxoSmithKline Biologicals: Food and Drug Administration 2009). Therefore, HPV vaccination can have potential cardiovascular benefits and public health implications.

In a further aspect, the presence of HPV can be used as a diagnostic or prognostic marker. Detecting the presence of HPV infection is useful in identifying and targeting subjects at risk for CVD who may require additional attention to avoid the development of cardiovascular events.

### I. ANTI-HUMAN PAPILLOMAVIRUS (HPV) AGENTS

In certain embodiments a subject is administered an HPV vaccine to treat or prevent CVD. A vaccine will comprise or produce one or more antigens that induce an immune response to HPV when administered to a subject. The inventors' analysis indicates that infection with cancer-associated HPVs (HPV 16, 18, and non-vaccine oncogenic types) has a strong association with CVD. An important step towards reducing the disabling impact of CVD is early identification of (and potential interventions for) potential risk factors.

Human papillomavirus (HPV) is a generic name for a group of epithelial cell specific viruses that have a diversity of DNA restriction enzyme profiles and different capsid protein antigenicity. Such viruses show similar shapes and are widespread in human and animals. They are highly specific, without cross-species transmission. There are more than 110 defined types of HPV. They are grouped into high-risk types and low-risk types, based on the correlation of the HPV types and oncogenic risks. Low-risk (non-oncogenic) type HPVs, e.g., HPV 6, 11, 42, 43, 44, often cause benign lesions, such as external genital warts. High-risk (oncogenic) type HPVs, e.g., HPV 16, 18, 31, 33, 35, 39, 45, 51, 52, 56, 58, 59, and 68, are associated with the development of cervical cancer and cervical intraepithelial neoplasm (CIN). Research on specimens from worldwide cervical cancer tissues indicated that HPV 16 and 18 have the highest infection rates. Among all of the detected types, HPV16 accounts for 50%, HPV18 for 14%, HPV45 for 8%, HPV31 for 5% and HPVs of other types account for 23% of infections. The HPV viral genome consists of three gene regions: early region (E), late region (L), and non-coding upstream regulatory region (URR), in which the early region comprises six early open reading frames, respectively defined as E1, E2, E4, E5, E6 and E7. Proteins E6 and E7 are oncoproteins, and play an important role in viral replication and immortalization and transformation of cells (Park et al., Cancer 76:1902-13, 1995). The late genes L1 and L2 are transcribed/translated and serve as structural proteins that encapsidate the amplified viral genomes.

An “oncogenic HPV strain” is an HPV strain that is known to cause cervical cancer as determined by the National Cancer Institute (NCI, 2001). “Oncogenic E6 proteins” are E6 proteins encoded by the above oncogenic HPV strains. The sequences of various HPV proteins are found as database entries at NCBI's GenBank database, as follows: HPV16-E6: GI:9627100; HPV18-E6: GI:9626069; HPV31-E6: GI:9627109; HPV35-E6: GI:9627127; HPV30-E6: GI:9627320; HPV39-E6: GI:9627165; HPV45-E6: GI:9627356; HPV51-E6: GI:9627155; HPV52-E6: GI:9627370; HPV56-E6: GI:9627383; HPV59-E6: GI:9627962; HPV58-E6: GI:9626489; HPV33-E6: GI:9627118; HPV66-E6: GI:9628582; HPV68b-E6: GI:I84383; HPV69-E6: GI:9634605; HPV26-E6: GI:396956; HPV53-E6: GI:9627377; HPV73: GI:1491692; HPV82: GI:9634614; HPV34 GI:396989; HPV67 GI:3228267; and HPV70 GI:1173493.

Examples of HPV vaccines and related compositions and methods are described in U.S. Pat. Nos. 5,820,870, 8,187,606, 8,101,342, 7,858,098, 7,758,866, 7,709,010, and 7,488,791, and US patent publications 20120164173, 20120107350, 20120093821, 20120053509, 20110189229, 20110177112, 20100189744, and 20100183648 each of which is incorporated herein by reference.

### II. CARDIOVASCULAR DISEASE (CVD)

Cardiovascular disease (CVD) is the general term for heart and blood vessel diseases, including atherosclerosis, coronary heart disease, cerebrovascular disease, aorto-iliac disease, and peripheral vascular disease. Subjects with CVD may develop a number of complications or sequelae, including, but not limited to, myocardial infarction, stroke, angina pectoris, transient ischemic attacks, congestive heart failure, aortic aneurysm, and death. CVD accounts for one in every two deaths in the United States and is the number one killer disease. Thus, prevention and treatment of cardiovascular disease is an area of major public health importance.

A low-fat diet and exercise are recommended to prevent CVD. In addition, a number of therapeutic agents may be prescribed by medical professionals to those individuals who are known to be at risk for developing or having CVD. These include lipid-lowering agents that reduce blood levels of cholesterol and trigylcerides, agents that normalize blood pressure, agents that prevent activation of platelets and decrease vascular inflammation (e.g., aspirin or platelet ADP receptor antatoginist (e.g., clopidogrel and ticlopidine)), and pleotrophic agents such as peroxisome proliferator activated receptor (PPAR) agonists, with broad-ranging metabolic effects that reduce inflammation, promote insulin sensitization, improve vascular function, and correct lipid abnormalities. More aggressive therapy, such as administration of multiple medications or surgical intervention may be used in those individuals who are at high risk. Since CVD therapies may have adverse side effects, it is desirable to have methods for identifying those individuals who are at risk, particularly those individuals who are at high risk, of developing or having CVD.

Currently, several risk factors are used by medical professionals to assess an individual's risk of developing or having CVD and to identify individuals at high risk. Major risk factors for cardiovascular disease include age, hypertension, family history of premature CVD, smoking, high total cholesterol, low HDL cholesterol, obesity, and diabetes. The major risk factors for CVD are additive, and are typically used together by physicians in a risk prediction algorithm to target those individuals who are most likely to benefit from treatment for CVD. These algorithms achieve a high sensitivity and specificity for predicting risk of CVD within 10 years. However, the ability of the present algorithms to predict a higher probability of developing CVD is limited. Among those individuals with none of the current risk factors, the 10-year risk for developing CVD is still about 2%. In addition, a large number of CVD complications occur in individuals with apparently low to moderate risk profiles, as determined using currently known risk factors. Thus, there is a need to expand the present cardiovascular risk algorithm to identify a larger spectrum of individuals at risk for or affected with CVD.

The mechanism of atherosclerosis is not well understood. Over the past decade a wealth of clinical, pathological, biochemical, and genetic data support the notion that atherosclerosis is a chronic inflammatory disorder. Acute phase reactants (e.g. C-reactive protein, complement proteins), sensitive but non-specific markers of inflammation, are enriched in fatty streaks and later stages of atherosclerotic lesions. In a recent prospective clinical trial, base-line plasma levels of C-reactive protein independently predicted risk of first-time myocardial infarction and stroke in apparently healthy individuals. U.S. Pat. No. 6,040,147 describes methods that use C-reactive protein, cytokines, and cellular adhesion molecules to characterize an individual's risk of developing a cardiovascular disorder. Although useful, these markers may be found in the blood of individuals with inflammation due to causes other than CVD, and thus, these markers may not be specific enough. Moreover, modulation of their levels has not been shown to predict a decrease in the morbidity or mortality of CVD.

The present invention provides methods for characterizing a subject's risk of having cardiovascular disease. The present invention also provides methods of characterizing a subject's risk of developing cardiovascular disease. In another embodiment, the present invention provides methods for characterizing a subject's risk of experiencing a complication of cardiovascular disease. The present methods are especially useful for identifying those subjects who are in need of CVD therapies.

The present invention also relates to methods of treating a subject to reduce the risk of a cardiovascular disorder or complication of such disorder. In one embodiment, the method comprises determining the presence or absence of HPV in a sample of a subject. In some embodiments, the HPV vaccine is administered after confirming that the sample does not contain HPV (i.e., the subject is HPV negative). In other embodiments, the subject can be administered one or more therapeutic CVD agents after confirming that the sample contains HPV (i.e., the subject is HPV positive). In other embodiments, the HPV vaccine is administered in combination (either co-administered or co-formulated) with one or more therapeutic CVD agents.

Therapeutic agents for CVD include, but are not limited to an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, a cyclooxygenase-2 inhibitor, an angiotensin system inhibitor, and/or combinations thereof. The agent is administered in an amount effective to lower the risk of the subject developing a future cardiovascular disorder.

“Anti-inflammatory” agents include but are not limited to, Aldlofenac; Aldlometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; and Zomepirac Sodium.

“Anti-thrombotic” and/or “fibrinolytic” agents include but are not limited to, Plasminogen (to plasmin via interactions of prekallikrein, kininogens, Factors XII, XIIIa, plasminogen proactivator, and tissue plasminogen activator (TPA)) Streptokinase; Urokinase: Anisoylated Plasminogen-Streptokinase Activator Complex; Pro-Urokinase; (Pro-UK); rTPA (alteplase or activase; r denotes recombinant); rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; retaplase; Trifenagrel; Warfarin; and Dextrans.

“Anti-platelet” agents include but are not limited to, Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine; and Anagrelide.

“Lipid-reducing” agents include but are not limited to, gemfibrozil, cholystyramine, colestipol, nicotinic acid, probucol lovastatin, fluvastatin, simvastatin, atorvastatin, pravastatin, cerivastatin, and other HMG-CoA reductase inhibitors.

“Direct thrombin inhibitors” include but are not limited to, hirudin, hirugen, hirulog, agatroban, PPACK, and thrombin aptamers.

“Glycoprotein Ith/IIIa receptor inhibitors” are both antibodies and non-antibodies, and include but are not limited to ReoPro (abcixamab), lamifiban, and tirofiban.

“Calcium channel blockers” are a chemically diverse class of compounds having important therapeutic value in the control of a variety of diseases including several cardiovascular disorders, such as hypertension, angina, and cardiac arrhythmias (Fleckenstein, Cir Res. 52(suppl. 1):13-16, 1983; Fleckenstein, Experimental Facts and Therapeutic Prospects, John Wiley, New York (1983); McCall, Curr Pract Cardiol 10:1-11, 1985). Calcium channel blockers are a heterogenous group of drugs that prevent or slow the entry of calcium into cells by regulating cellular calcium channels. (Remington, The Science and Practice of Pharmacy, Nineteenth Edition, Mack Publishing Company, Eaton, Pa., p. 963 (1995)). Most of the currently available calcium channel blockers belong to one of three major chemical groups of drugs, the dihydropyridines, such as nifedipine, the phenyl alkyl amines, such as verapamil, and the benzothiazepines, such as diltiazem. Other calcium channel blockers include, but are not limited to, anrinone, amlodipine, bencyclane, felodipine, fendiline, flunarizine, isradipine, nicardipine, nimodipine, perhexylene, gallopamil, tiapamil and tiapamil analogues (such as 1993RO-11-2933), phenyloin, barbiturates, and the peptides dynorphin, omega-conotoxin, and omega-agatoxin, and the like and/or pharmaceutically acceptable salts thereof.

“Beta-adrenergic receptor blocking agents” are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, beftunolol, betaxolol, bunitrolol, carteolol, celiprolol, hydroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hydroxypropoxy)-3-pyridenecarbonitrilHCl, 1-butylamino-3-(2,5-dichlorophenoxy-)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, and 7-(2-hydroxy-3-t-butylaminpropoxy)phthalide. The above-identified compounds can be used as isomeric mixtures, or in their respective levorotating or dextrorotating form.

Suitable COX-2 inhibitors include, but are not limited to, COX-2 inhibitors described in U.S. Pat. Nos. 5,474,995; 5,521,213; 5,536,752; 5,550,142; 5,552,422; 5,604,253; 5,604,260; 5,639,780; 5,643,933; 5,677,318; 5,691,374; 5,698,584; 5,710,140; 5,733,909; 5,789,413; 5,817,700; 5,849,943; 5,861,419; 5,922,742; and 5,925,631, each of which is incorporated herein by reference.

An “angiotensin system inhibitor” is an agent that interferes with the function, synthesis, or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na⁺ in plasma tend to activate the system, while factors that increase these parameters tend to suppress its function. Such inhibitors are well known to those of ordinary skill in the art. Examples of classes of such compounds include antibodies (e.g., to renin), amino acids and analogs thereof (including those conjugated to larger molecules), peptides (including peptide analogs of angiotensin and angiotensin I), pro-renin related analogs, etc. Among the most potent and useful renin-angiotensin system inhibitors are renin inhibitors, ACE inhibitors, and angiotensin II antagonists.

Examples of angiotensin II antagonists include: peptidic compounds (e.g., saralasin, [(San1)(Val5)(Ala8)] angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1):1-7, 1988); 4,5,6,7-tetrahydro-1H-imidazo[4,5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP patent 253,310); ES8891 (N-morpholinoacetyl-(−1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl) methyl]1H-imidazole-5-yl[methylane]-2-thiophenepropanoic acid, Smith Kline Beecham Pharmaceuticals, Pa.); Losartan (DUP7531MK954, DuPont Merck Pharmaceutical Company); Remikirin (RO42-5892, F. Hoffman LaRoche AG); and A2 agonists (Marion Merrill Dow) and certain non-peptide heterocycles (G. D. Searle and Company). Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Examples of renin inhibitors that are the subject of United States patents are as follows: urea derivatives of peptides (U.S. Pat. No. 5,116,835); amino acids connected by nonpeptide bonds (U.S. Pat. No. 5,114,937); di and tri peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437). All of cited patents are incorporated herein by reference.

### III. EXAMPLES

The following examples are included to demonstrate preferred embodiments of the invention. It should be appreciated by those of skill in the art that the techniques disclosed in the examples represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Association of HPV Infection and Cardiovascular Disease (CVD)

A. Results

Women in this study were generally young with a mean age of 37.9 years. The prevalence of CVD was low with a total of 60 women (2.5%) reported having either myocardial infarction or stroke. Among study participants (N=2450), 1141 women (46.6%) were positive for HPV DNA. Five hundred and seventy three women (23.2% of study population) had cancer-associated HPV types. Characteristics of the study sample by HPV genotypes are summarized in Table 1. Women without HPV infection tended to be non-smoker and to drink less, while women with cancer-associated HPV types were more likely to be younger and tended to have higher prevalence of CVD. There was no difference in prevalence of diabetes mellitus, lung disease, liver disease, thyroid disease, and cancer between groups.

TABLE 1
Characteristics of Study Participants According
to HPV Genotypes, NHANES 2003 to 2006 (N = 2,450)

| | Cancer- | | | | | | | | |
|---|---|---|---|---|---|---|---|---|---|
| | Associated | | Other | | | | | | |
| | HPV Types | | HPV Types | | Negative | | Total | | |
| Variables | (n = 573) | | (n = 568) | | (n = 1,309) | | (N = 2,450) | | p Value |
| Age group | | | | | | | | | <0.001 |
| 20-29 yrs | 218 | (38.0) | 141 | (24.8) | 355 | (27.1) | 714 | (29.2) | |
| 30-45 yrs | 209 | (36.5) | 255 | (44.9) | 546 | (41.7) | 1,010 | (41.2) | |
| 46-59 yrs | 146 | (25.5) | 172 | (30.3) | 408 | (31.2) | 726 | (29.6) | |
| Race | | | | | | | | | _0.001 |
| Mexican American | 113 | (19.7) | 114 | (20.1) | 279 | (21.3) | 506 | (20.7) | |
| Other Hispanics | 18 | (3.2) | 22 | (3.9) | 49 | (3.7) | 89 | (3.6) | |
| Non-Hispanic white | 254 | (44.3) | 242 | (42.6) | 691 | (52.8) | 1,187 | (48.4) | |
| Non-Hispanic black | 166 | (29.0) | 170 | (29.9) | 222 | (17.0) | 558 | (22.8) | |
| All others | 22 | (3.8) | 20 | (3.5) | 68 | (5.2) | 1,10 | (4.5) | |
| ≧12 alcoholic drinks | 382 | (66.7) | 363 | (63.9) | 785 | (60.0) | 1,530 | (62.5) | 0.016 |
| in the last yr | | | | | | | | | |
| Nonsmoker | 395 | (68.9) | 415 | (73.1) | 1,078 | (82.4) | 1,888 | (77.1) | <0.001 |
| Ever had sex behavior | 548 | (95.6) | 540 | (95.1) | 1,219 | (93.1) | 2,307 | (94.2) | 0.058 |
| Cardiovascular disease | 20 | (3.5) | 19 | (3.35) | 21 | (1.6) | 60 | (2.5) | 0.015 |
| Hypertension | 124 | (21.6) | 161 | (28.4) | 289 | (22.1) | 574 | (23.5) | 0.007 |
| Diabetes mellitus | 38 | (6.6) | 42 | (7.4) | 95 | (7.3) | 175 | (7.1) | 0.858 |
| Lung disease | 119 | (20.8) | 124 | (21.8) | 240 | (18.3) | 483 | (19.7) | 0.167 |
| Liver disease | 15 | (2.6) | 18 | (3.2) | 30 | (2.3) | 63 | (2.6) | 0.543 |
| Thyroid disease | 68 | (11.9) | 60 | (10.6) | 154 | (11.8) | 282 | (11.5) | 0.721 |
| Cancer | 33 | (5.8) | 28 | (4.9) | 68 | (5.2) | 129 | (5.3) | 0.810 |
| Cervical cancer | 12 | (2.1) | 10 | (1.8) | 22 | (1.7) | 44 | (1.8) | 0.822 |
| All other cancer | 21 | (3.7) | 18 | (3.2) | 46 | (3.5) | 85 | (3.5) | 0.893 |
| Sexually transmitted | 84 | (14.7) | 86 | (15.1) | 107 | (8.2) | 277 | (11.3) | <0.001 |
| disease | | | | | | | | | |

Cancer-associated human papillomavirus (HPV) types—HPV types 16, 18, 31, 33, 35, 39, 45 51, 52, 56, 58, 59, and 68.

Association Between HPV and Various Metabolic Risks.

Various metabolic risks, including systolic blood pressure, diastolic blood pressure, abdominal circumference, levels of blood glucose, triglyceride, and HDL cholesterol, and a composite metabolic Z score were compared in three different groups: cancer-associated HPV types, other HPV types, and negative HPV. The inventors did not find any association between HPV and various metabolic risks. Adjusted means of various metabolic risks were comparable across 3 different groups after controlling for age, race, and health/sex behaviors (Table 2).

TABLE 2
Relation of HPV to Various Metabolic Risks

| Metabolic Risks | HPV Genotypes | β*(Standard Error) | p Value | Adjusted Means† |
|---|---|---|---|---|
| Systolic blood | HPV negative | Reference | | 115.2 |
| pressure, mm Hg | Other HPV types | 0.92 (0.74) | 0.214 | 116.2 |
| | Cancer-associated | 0.62 (0.75) | 0.404 | 115.9 |
| | HPV types | | | |
| Diastolic blood | HPV negative | Reference | | 68.7 |
| pressure, mm Hg | Other HPV types | 0.95 (0.55) | 0.084 | 69.7 |
| | Cancer-associated | −0.34 (0.55) | 0.539 | 68.4 |
| | HPV types | | | |
| Abdominal | HPV negative | Reference | | 95.0 |
| circumference, cm | Other HPV types | 0.56 (0.81) | 0.494 | 95.5 |
| | Cancer-associated | 0.53 (0.82) | 0.513 | 95.5 |
| | HPV types | | | |
| Blood glucose, | HPV negative | Reference | | 92.9 |
| mg/dl | Other HPV types | −1.43 (1.31) | 0.275 | 91.4 |
| | Cancer-associated | −1.12 (1.32) | 0.396 | 91.7 |
| | HPV types | | | |
| Triglyceride, mg/dl | HPV negative | Reference | | 131.4 |
| | Other HPV types | −5.50 (7.93) | 0.488 | 125.9 |
| | Cancer-associated | −2.04 (8.01) | 0.799 | 129.4 |
| | HPV types | | | |
| HDL cholesterol, | HPV negative | Reference | | 60.2 |
| mg/dl | Other HPV types | −0.42 (0.85) | 0.617 | 59.8 |
| | Cancer-associated | −0.09 (0.85) | 0.912 | 60.2 |
| | HPV types | | | |
| Metabolic z-score | HPV negative | Reference | | _0.054 |
| | Other HPV types | 0.05 (0.14) | 0.729 | _0.006 |
| | Cancer-associated | 0.02 (0.14) | 0.898 | _0.036 |
| | HPV types | | | |

Cancer-associated HPV types—HPV types 16, 18, 31, 33, 35, 39, 45 51, 52, 56, 58, 59, and 68.
*The β coefficients can be interpreted as differences in various metabolic risks comparing women with different categories of HPV (human papillomavirus) genotypes to those with negative HPV.
†Adjusted for age, race, health behaviors (smoking and alcohol consumption), and sex behavior.

Association Between HPV and Cardiovascular Disease.

The results of multiple logistic regression analysis for the association between HPV and CVD were summarized in Table 2. After adjusting for age and race, presence of vaginal HPV DNA was associated with CVD. Odds ratio (OR) of CVD comparing women with presence of vaginal HPV DNA to those without was 2.46 (95% confidence interval [CI] 1.42-4.27). Further covariates adjustment, including health/sex behaviors, medical co-morbidities, composite metabolic Z score, and cardiovascular risk factors and management (Model 2 to Model 5), only mildly attenuated the association between HPV and CVD (Table 3). The inventors then examined the relation of HPV genotypes with CVD and found that cancer-associated HPV types were specifically associated with either myocardial infarction or stroke. After controlling for demographics, health/sex behaviors, medical co-morbidities, and cardiovascular risk factors and management, OR of CVD comparing women with cancer-associated HPV types to those with negative HPV were 2.86 (95% CI1.43-5.70). Presence of other types of HPV DNA was not associated with CVD in the multivariate Models 2 to 5. Interactive effects between age and HPV genotypes on CVD were tested across Model 1 to Model 5 and no interaction was identified.

TABLE 3
Association Between Cardiovascular Disease and HPV

| | Model 1 | | Model 2 | | Model 3 | | Model 4 | | Model 5 | |
|---|---|---|---|---|---|---|---|---|---|---|
| | | p | | p | | p | | p | | p |
| | OR (95% CI) | Value | OR (95% CI) | Value | OR (95% CI) | Value | OR (95% CI) | Value | OR (95% CI) | Value |
| Presence of HPV | | | | | | | | | | |
| DNA | | | | | | | | | | |
| Negative | 1.0 | | 1.0 | | 1.0 | | 1.0 | | 1.0 | |
| (reference) | | | | | | | | | | |
| Positive | 2.46 (1.42-4.27) | 0.001 | 2.13 (1.21-3.74) | 0.009 | 2.14 (1.21-3.79) | 0.009 | 2.30 (1.27-4.16) | 0.006 | 2.06 (1.14-3.74) | 0.017 |
| HPV genotypes | | | | | | | | | | |
| Negative HPV | 1.0 | | 1.0 | | 1.0 | | 1.0 | | 1.0 | |
| (reference) | | | | | | | | | | |
| Other HPV types | 2.13 (1.12-4.06) | 0.021 | 1.91 (0.99-3.69) | 0.055 | 1.88 (0.97-3.67) | 0.063 | 1.89 (0.94-3.78) | 0.072 | 1.70 (0.84-3.43) | 0.137 |
| Cancer-associated | 2.87 (1.52-5.43) | 0.001 | 2.39 (1.24-4.61) | 0.009 | 2.46 (1.27-4.77) | 0.008 | 2.86 (1.43-5.70) | 0.003 | 2.53 (1.27-5.01) | 0.008 |
| HPV types | | | | | | | | | | |

B. Methods

Data Source and Study Sample.

The data comes from the NHANES 2003-2006, a population-based cross-sectional survey designed to collect information on the health and nutrition of the US civilian non-institutionalized population. Detailed Survey Operations Manuals, Consent Documents, and Brochures of the NHANES 2003-2006 are available on the NHANES website accessible on the world wide web at cdc.gov/nchs/nhanes.htm.

Of 3467 females aged 20 to 59 years who were interviewed at home for the 2003-2006 cycles, 3330 were examined in a mobile examination center. Among them, 2829 females (85.0%) submitted cervicovaginal swab specimens while 501 females did not. Of the 2829 females who submitted cervicovaginal swab specimens, 42 females submitted an inadequate swab specimen. Five hundred and forty three females (16.3%) were considered non-responders because they either submitted an inadequate swab specimen (n=42) or they did not submit a swab specimen (n=501). Compared to responders, non-responders were significantly more likely to be of all other race (race/ethnicity other than Hispanics, Black, or White) (8.8% vs 4.4%, p<0.001), and to be younger (34.0 vs 37.7 years, p<0.001). Among the 2787 women with adequate specimen, 337 participants were further excluded because of missing covariates in laboratory measurements or health behavior questionnaire, leaving 2450 women as the final analytic sample.

HPV DNA Examination and Genotyping.

Self-collected vaginal swabs were evaluated for HPV infection. Vaginal specimens were processed, stored and shipped to Atlanta, Ga. for analysis. DNA was extracted using modifications of QIAmp Mini Kit protocol within one month of sample collection, and HPV detection and genotyping were conducted as described elsewhere (Dunne et al. JAMA 297(8):813-9, 2007). In brief, HPV detection and typing were performed by using the Roche Linear Array Assay (Roche Molecular Systems Inc., Pleasanton, Calif.). The assay used HPV L1 consensus polymerase chain reaction (PCR) with biotinylated PGMY09/11 primer sets and β-globin as an internal control for sample amplification. The primer mix amplifies essentially all HPV types found in the genital tract. After amplification the samples were then typed by hybridization to the typing strips followed by colorimetric detection. The strip was a linear array of probes specific for 37 HPV types (6, 11, 16, 18, 26, 31, 33, 35, 39, 40, 42, 45, 51, 52, 53, 54, 55, 56, 58, 59, 61, 62, 64, 66, 67, 68, 69, 70, 71, 72, 73, 81, 82, 83, 84, IS39, and 89) and for the positive β-globin as well. Types were read by comparing the reaction pattern to the typing template. Samples negative for both β-globin and HPV were considered inadequate for interpretation (n=42, 1.48%) and were omitted from further analysis. HPV types 16, 18, 31, 33, 35, 39, 45, 51, 52, 56, 58, 59, and 68 were defined as oncogenic or cancer-associated HPV types (Bosch et al. J Natl Cancer Inst 87(11):796-802, 1995).

Cardiovascular Disease (CVD).

CVD was ascertained by self-reported questionnaires: “Has a doctor or other health professional ever told you that you had a heart attack (also called myocardial infarction)?” or “Has a doctor or other health professional ever told you that you had a stroke?” Answering yes to either question was coded positive for CVD.

Assessment of Demographics and Clinical Factors.

Age, race, sex behavior, and alcohol consumption were obtained by self-report. Sex was defined as vaginal, oral, or anal sex. Body mass index (BMI), calculated as weight in kilograms divided by the square of height in meter, was categorized according to the National Institutes of Health obesity standards: <18.5=underweight, 18.5-24.9=normal weight, 25.0-29.9=overweight, and >30=obese (National Heart, Lung, and Blood Institute. Obesity Education Initiative: The Evidence Report. Bethesda, Md.: Nation Institutes of Health; 1998). Using their serum cotinine concentrations (ng/mL), we classified smoking status of participants in four groups: nonsmoker (<14), light smoker (14-99), moderate smoker (100-199), and heavy smoker (≧200) (Wei et al. Am J Public Health 91(2):258-64, 2001). Co-morbidities including lung disease (defined as asthma, emphysema or chronic bronchitis), liver disease, thyroid disease, sexually transmitted diseases (STDs), or cancer (including cervical cancer or all other cancer), use of cholesterol-lowering medications, and use of anti-hypertensive medications were ascertained by self-report. Diabetes was defined by self-report of a physician's diagnosis, the presence of a fasting (fasting ≧6 hours) plasma glucose level ≧126 mg/dL or a non-fasting (fasting <6 hours) glucose level ≧200 mg/dL, hemoblogin A_{1c} (A1C)≧6.5% (5), or the use of diabetic medications. Hypertension was defined as mean systolic BP≧140 mmHg, mean diastolic BP≧90 mmHg, physician diagnosis, or use of anti-hypertensive medications. Mean BP was composed of up to 4 readings on 2 separate occasions. Waist circumference was measured at the iliac crest to the nearest 0.1 cm. Serum triglycerides, high-density lipoprotein (HDL) cholesterol, C-reactive protein (CRP), serum creatinine, urinary albumin, urinary creatinine were analyzed by laboratory methods reported elsewhere (National Health and Nutrition Examination Survey 2003-2004 Lab Methods; National Health and Nutrition Examination Survey 2005-2006 Lab Methods). Urinary albumin-to-creatinine ratio (UACR, in mg/g) was calculated accordingly. Estimated glomerular filtration rate (eGFR) was calculated by using the Modification of diet in renal disease (MDRD) study equation (Levey et al. Ann Intern Med 130(6):461-70, 1999).

Analysis.

Demographic and clinical characteristics of the study population were presented according to presence of HPV genotypes, namely, cancer-associated HPV types (N=573), other HPV types (N=568), or negative HPV (N=1309). Chi-squared test or t-test was used to determine differences between groups. Association of metabolic risks with HPV genotypes was examined using multiple regression. To consider various metabolic risk factors as a whole, we also calculated the metabolic Z score. The score was derived by converting each component of the metabolic risk factors, namely blood pressure, waist circumference, levels of glucose, triglyceride level, and HDL level, into a Z score based on means of the study population (Lin et al. Diabetes Care 31(5):1015-20, 2008). The metabolic Z score was calculated by summation of the former four Z scores minus the HDL Z score. Multiple logistic regression analysis was used to examine the association of HPV DNA, HPV genotypes and CVD. Women with negative HPV DNA were the reference category. We used an extended-model approach for covariate adjustment: Model 1=Age and race; Model 2=Model 1+health behaviors (smoking and alcohol consumption) and sex behavior; Model 3=Model 2+medical co-morbidities (lung disease, liver disease, thyroid disease, eGFR, 20 STDs, history of cervical cancer, history of all other cancer); Model 4=Model 3+cardiovascular risk factors and management (hypertension, diabetes mellitus, waist circumference, triglycerides, HDL cholesterol, log-transformed levels of CRP and UACR, use of anti-hypertensives or cholesterol-lowering medications); and Model 5=Model 3+metabolic Z score, use of anti-hypertensives or cholesterol-lowering medications.

NHANES weights were not adjusted in the analyses because they apply to prevalence estimates of the entire population and the study aims to evaluate associations in a certain subset of women with valid data of HPV DNA examination and genotyping. Data analyses were performed using STATA 10.0 software (STATA Corporation, College Station, Tex., USA).

### Example 2

### Clinical Trial Design

It is contemplated that HPV vaccine protects patients against atherosclerosis and its complications.

Methods:

Patients (both males and females) who are younger than 60 years of age and presents with acute coronary syndrome to hospitals who do not have HPV infection are randomized 1:5 fashion to (a) HPV vaccine or (b) placebo, assuming that at least 20% of the enrolled patients will acquire HPV during the study period.

Base line laboratory tests are obtained: high-sensitivity CRP, carotid artery intimal thickness, lipid profile, hemoglobin A1C, ankle-brachial index (ABI), EKG, base-line echocardiogram, complete blood chemistry, complete blood count, and others.

The enrolled patients are followed for 5 years.

The enrolled patients will visit the study clinics twice a year to have HPV status determined.

The primary composite cardiovascular endpoints (death from cardiovascular causes, non-fatal myocardial infarction, and non-fatal stroke) are examined in these two groups.

The secondary endpoints include: (i) Carotid artery intimal thickness (evaluated every year by ultra-sensitive carotid ultrasound) (ii) High-sensitivity CRP (iii) Clinical markers for atherosclerosis other than strokes and myocardial infarctions including claudication, aneurysm formation, angina pectoris, and others.

The study (HPV vaccine) group is expected to have a lower rate of the primary/secondary composite cardiovascular endpoints than does the control group.

The HPV positive group (a portion of the control group) is expected to have a higher rate of the primary/secondary composite cardiovascular endpoints than dose the HPV negative group (the remainder of the control group and the vast majority of the study group).

1. A method for treating or preventing cardiovascular disease (CVD) in a subject comprising administering an HPV vaccine to a subject that is at least 27 years old thereby reducing the likelihood of developing CVD or its sequelae.

2. The method of claim 1, wherein the subject is at least 45 years old.

3. The method of claim 1, wherein the vaccine induces immunity to an oncogenic HPV.

4. The method of claim 3, wherein the HPV vaccine induces immunity to HPV16.

5. The method of claim 3, wherein the HPV vaccine induces immunity to HPV 18.

6. The method of claim 1, wherein the HPV vaccine induces an immune response to one or more of L1, L2, E1, E2, E3, E4, E5, E6, E7 and E8 proteins of HPV.

7. The method of claim 6, wherein the HPV vaccine induces an immune response to an HPV L1 protein.

8. The method of claim 1, wherein the vaccine induces immunity to a non-oncogenic HPV.

9. The method of claim 1, wherein CVD is atherosclerosis, unstable angina, chronic stable angina, sudden cardiac death, aneurysm formation, intermittent claudication, or renal artery stenosis.

10. The method of claim 1, wherein CVD is atherosclerosis.

11. The method of claim 1, wherein the CVD sequela is myocardial infarction.

12. The method of claim 1, wherein the CVD sequela is stroke.

13. The method of claim 1, wherein the subject is female.

14. The method of claim 1, wherein the subject has hypertension, has a family history of premature CVD, smokes, has high total cholesterol, has low HDL cholesterol, is obese, or has diabetes.

15. The method of claim 1, further comprising detecting the presence or absence of HPV in the subject prior to administering the HPV vaccine.
